(19) United States
(12) Patent Application Publication
(10) Pub. No.: US 2021/0220232 A1
(43) Pub. Date: Jul. 22, 2021

(54) OIL-IN-WATER TYPE EMULSION COSMETIC USING PARTIALLY CROSSLINKED OR CROSSLINKED DIMETHYLPOLYSILOXANE

(71) Applicant: Shiseido Company, Ltd., Tokyo (JP)

(72) Inventors: Ryo FURUKAWA, Tokyo (JP); Shigeo TAKAHASHI, Tokyo (JP)

(73) Assignee: Shiseido Company, Ltd., Tokyo (JP)

(21) Appl. No.: 16/972,219

(22) PCT Filed: May 29, 2019
(86) PCT No.: PCT/JP2019/021371

§ 371 Date: Dec. 4, 2020

(30) Foreign Application Priority Data
Jun. 6, 2018 (JP) 2018-109058

(51) Int. Cl. A61K 8/06; A61K 8/891; A61K 8/34; A61K 8/46; A61K 8/92; A61Q 19/00

(52) U.S. Cl. CPC A61K 8/062; A61K 8/891; A61Q 19/00; A61K 8/46; A61K 8/92; A61K 8/34

(57) ABSTRACT

An oil-in-water emulsion cosmetic comprising: an anionic surfactant (A) having a sulfonic acid group in a molecule, a partially-crosslinked or crosslinked dimethylpolysiloxane (B), an animal/plant-derived or chemically synthesized oily component (C) that is solid at 25° C., and a silicone oil (D) having a kinematic viscosity at 25° C. of 100 cSt or less.

### CROSS REFERENCE TO RELATED APPLICATIONS

The present application is based upon and claims the benefit of the priority of Japanese Patent Application No. 2018-109058, filed on Jun. 6, 2018, the disclosure of which is incorporated herein in its entirety by reference.

### TECHNICAL FIELD

The present invention relates to an oil-in-water emulsion cosmetic using a partially-crosslinked or crosslinked dimethylpolysiloxane, and particularly to an improvement in stability of the cosmetic at low viscosity and an improvement in usability.

### BACKGROUND ART

As methods for preparing emulsions that are free of stickiness or sliminess upon application to skin, the following methods are known conventionally: a method of emulsifying a small amount of an oil agent by a surfanctant; a method of emulsifying by a water-soluble polymer such as an alkyl-modified carboxyvinyl polymer; a method of using both; and the like. Moreover, an oil-in-water emulsion composition in which a higher alcohol, an acylsulfonate-type anionic surfactant and a polar oil are combined is reported to be have good stability and is excellent in feeling in use such as non-stickiness (Patent Literature 1).

Moreover, it is reported that an oil-in-water emulsion skin cosmetic that achieved both of feeling in use and storage stability is obtained by blending a crosslinked methylpolysiloxane to an oil agent comprising a silicone oil of low viscosity, and emulsifying by a combination of a POE addition type nonionic surfactant having an HLB of 10 or greater and a nonionic surfactant having an HLB of 6 or less (Patent Literature 2).

However, problems of stickiness or sliminess could not be solved sufficiently with the above-mentioned preparation methods. Moreover, when a partially-crosslinked or crosslinked dimethylpolysiloxane was blended to improve feeling in use, aggregation occurred in a cosmetic of low viscosity, and thus it was a problem that storage stability was poor.

### CITATION LIST

### Patent Literatures

Patent Literature 1: Japanese Unexamined Patent Publication No. 2008-044866 A.

Patent Literature 2: Japanese Unexamined Patent Publication No. 2011-126810 A.

### SUMMARY OF INVENTION

### Technical Problem

The present invention has been made in view of the above-mentioned conventional art, and an object to be solved is to improve stability at low viscosity in an oil-in-water emulsion cosmetic using a partially-crosslinked or crosslinked dimethylpolysiloxane, and to provide an oil-in-water emulsion cosmetic having excellent feeling in use.

### Solution to Problem

The present inventors have diligently studied the above-mentioned problem, and as a result, they have found that an oil-in-water emulsion cosmetic that has an excellent stability over time at low viscosity and an excellent feeling in use can be obtained by blending, under predetermined conditions: an anionic surfactant (A) having a sulfonic acid group in a molecule; a partially-crosslinked or crosslinked dimethylpolysiloxane (B); an animal/plant-derived or chemically synthesized oily component (C) that is solid at 25° C.; and a silicone oil (D) having a kinematic viscosity at 25° C. of 100 cSt or less.

That is, the oil-in-water emulsion cosmetic according to the present invention comprises the following components (A) to (D) and satisfies the following conditions (1) to (3).

(A) An anionic surfactant having a sulfonic acid group represented by the following general formula in a molecule.

R₁CO-a-(CH₂)nSO₃M₁ (I)

[In the formula (I): R1CO— represents a saturated or unsaturated fatty acid residue (acyl group) having 10 to 22 carbon atoms on average; a represents —O— or —NR2- (R2 represents a hydrogen atom or a C₁₋₃ alkyl group); M1 represents a hydrogen atom, alkaline metal, alkaline earth metal, ammonium, or organic amine; and n represents an integer of 1 to 3.]

(B) A partially-crosslinked or crosslinked dimethylpolysiloxane
(C) An animal/plant-derived or chemically synthesized oily component that is solid at 25° C.
(D) A silicone oil having a kinematic viscosity at 25° C. of 100 cSt or less

### Condition (1): (A)/((B)+(C)+(D))=0.01 to 1.0

### Condition (2): (A)/(C)=0.01 to 1.0

Condition (3): viscosity at 25° C. being 10 to 100000 mPa·s

In the emulsion cosmetic, it is preferred that (A) is one type or two or more types selected from stearoyl methyl taurate, cocoyl methyl taurate and lauroyl methyl taurate.

In the emulsion cosmetic, it is preferred that a silicone surfactant (E) is further comprised.

In the emulsion cosmetic, it is preferred that 0.05 to 2.0% by mass of the silicone surfactant (E) is comprised in the cosmetic.

In the emulsion cosmetic, it is preferred that (D) has a phenyl group in a molecule.

In the emulsion cosmetic, it is preferred that 0.1 to 20% by mass of (D) is comprised in the cosmetic.

In the emulsion cosmetic, it is preferred that a polyol or a sugar alcohol having Log P of −1.0 to −3.0 is comprised.

In the emulsion cosmetic, it is preferred that an ester oil or an animal/vegetable oil having an IOB value in an organic conceptual diagram of 0 to 0.2 is comprised.

In the emulsion cosmetic, it is preferred that an ester oil that has an IOB value in an organic conceptual diagram of 0 to 0.2 and is liquid at 25° C. is comprised.

In the emulsion cosmetic, it is preferred that (C) is a C₁₀₋₂₄ aliphatic alcohol having a linear or branched chain.

In the emulsion cosmetic, it is preferred that (C) is behenyl alcohol.

In the emulsion cosmetic, it is preferred that (E) is a polyglyceryl modified silicone.

In the emulsion cosmetic, it is preferred that (E) is a polyether (3-40) modified polysiloxane.

In the emulsion cosmetic, it is preferred that a water-soluble thickener (F) is comprised.

In the emulsion cosmetic, it is preferred that a semi low-soluble or low-soluble drug having a Log P value of 10 to 30 is comprised.

In the emulsion cosmetic, it is preferred that the semi low-soluble or low-soluble drug having a Log P value of 10 to 30 is selected from Coenzyme Q10, stearyl glycyrrhetinate, astaxanthin, tranexamic acid, allantoin, alkoxy salicylic acid and a whitening agent (other components).

In the emulsion cosmetic, it is preferred that (B) is dimethicone/phenyl vinyl dimethicone crosspolymer, polysilicone-11 or a crosslinked methylpolysiloxane.

### Advantageous Effects of Invention

According to the present invention, it was found that an oil-in-water emulsion cosmetic having an excellent stability over time at low viscosity and an excellent feeling in use can be obtained by blending, under predetermined conditions, an anionic surfactant (A) having a sulfonic group in a molecule, a partially-crosslinked or crosslinked dimethylpolysiloxane (B), an animal/plant-derived or chemically synthesized oily component (C) that is solid at 25° C., and a silicone oil (D) having a kinematic viscosity at 25° C. of 100 cSt or less.

### DESCRIPTION OF EMBODIMENTS

The oil-in-water emulsion cosmetic according to the present invention comprises the following components (A) to (D) and satisfies the following conditions (1) to (3).

(A) An anionic surfactant having a sulfonic acid group represented by a general formula (I) in a molecule
(B) A partially-crosslinked or crosslinked dimethylpolysiloxane
(C) An animal/plant-derived or chemically synthesized oily component that is solid at 25° C.
(D) A silicone oil having a kinematic viscosity at 25° C. of 100 cSt or less

### Condition (1): (A)/((B)+(C)+(D))=0.01 to 1.0

### Condition (2): (A)/(C)=0.01 to 1

Condition (3): viscosity at 25° C. being 10 to 100000 mPa·s

### (A) Anionic Surfactant Having a Sulfonic Acid Group in a Molecule

The anionic surfactant having a sulfonic acid group in a molecule used in the present invention is represented by the following general formula (I).

[Formula 1]

R₁CO-a-(CH₂)nSO₃M₁ (I)

In the above-identified formula (I): R₁CO— represents a saturated or unsaturated fatty acid residue (acyl group) having 10 to 22 carbon atoms on average; a represents —O— or —NR₂— (R₂ represents a hydrogen atom or a C₁₋₃ alkyl group); M₁ represents a hydrogen atom, alkaline metal, alkaline earth metal, ammonium, or organic amine; and n represents an integer of 1 to 3.

When the anionic surfactant (A) having a sulfonic acid group in a molecule of the present invention is used in the oil-in-water emulsion cosmetic, it is preferably 0.1 to 1.5% by mass in the cosmetic. When it exceeds 1.5% by mass, stickiness may be imparted. When it is less than 0.1% by mass, emulsion stability may deteriorate.

The anionic surfactant having a sulfonic acid group in a molecule that can be used in the present invention is not limited in particular; however, stearoyl methyl taurate, cocoyl methyl taurate and lauroyl methyl taurate are preferred.

Examples of commercially available products of the anionic surfactant having a sulfonic acid group in a molecule include: NIKKOL SMT (sodium methyl stearoyl taurate, manufactured by Nikko Chemicals Co., Ltd.); NIKKOL LMT (sodium methyl lauroyl taurate, manufactured by Nikko Chemicals Co., Ltd.); NIKKOL CMT-30 (sodium methyl cocoyl taurate, manufactured by Nikko Chemicals Co., Ltd.); and the like.

### (B) Dimethylpolysiloxane

Dimethylpolysiloxane (B) used in the present invention is partially-crosslinked or crosslinked.

When the partially-crosslinked or crosslinked dimethylpolysiloxane (B) of the present invention is used in the oil-in-water emulsion cosmetic, it is preferably 0.1 to 2% by mass in the cosmetic, and more preferably 0.1 to 0.8% by mass. When it exceeds 2% by mass, aggregation of the partially-crosslinked or crosslinked dimethylpolysiloxane may occur. When it is less than 0.1% by mass, smooth and good texture upon use may not be achieved.

The partially-crosslinked or crosslinked dimethylpolysiloxane (B) of the present invention is not limited in particular; however, alkyl-crosslinked polydimethylsiloxane, polysilicone-11, crosslinked methylpolysiloxane, dimethicone/phenyl vinyl dimethicone crosspolymer, crosslinked polyether-modified silicone, and PEG-15/lauryl polydimethylsiloxyethyl dimethicone crosspolymer are preferred.

Among the above, dimethicone/phenyl vinyl dimethicone crosspolymer, polysilicone-11, and crosslinked methylpolysiloxane are superior.

Examples of commercially available products of the partially-crosslinked or crosslinked dimethylpolysiloxane (B) include: DOW CORNING® 9041 SILICONE ELASTOMER BLEND (dimethicone crosspolymer dimethicone, manufactured by Dow Corning Toray Co., Ltd.); Gransil DMG-3 (Dimethicone (and) Polysilicone-11, manufactured by Grant Industries, Inc.); KSG-210 (dimethicone/(PEG-10/15) crosspolymer, manufactured by Shin-Etsu Chemical Co., Ltd.); and the like.

### (C) Animal/Plant-Derived or Chemically Synthesized Oily Component

The animal/plant-derived or chemically synthesized oily component used in the present invention is solid at 25° C. When it is not solid, aggregation of the partially-crosslinked or crosslinked dimethylpolysiloxane may occur, and thus it is not preferred.

When the animal/plant-derived or chemically synthesized oily component (C) of the present invention is used in the oil-in-water emulsion cosmetic, it is preferably 0.5% to 10% by mass in the cosmetic. When it exceeds 10%, smooth and good texture upon use may not be achieved. When it is less than 0.5%, aggregation of the partially-crosslinked or crosslinked dimethylpolysiloxane may occur.

The animal/plant-derived or chemically synthesized oily component that can be used in the present invention is not limited in particular; however, a C₁₀₋₂₄ aliphatic alcohol having a linear or branched chain, sphingosine, an insulator, ceramide, and the like are preferred.

Examples of these alcohols include: lauryl alcohol; cetyl alcohol; stearyl alcohol; behenyl alcohol; myristyl alcohol; oleyl alcohol; cetostearyl alcohol; monostearyl glycerine ether (batyl alcohol); 2-decyltetradecanol; lanolin alcohol; cholesterol; hexyldodecanol; isostearyl alcohol; octyldodecanol; and the like.

Among the above, behenyl alcohol, sphingosine, and ceramide are superior.

Examples of commercially available products of the animal/plant-derived or chemically synthesized oily component (C) include: NIKKOL Behenyl Alcohol 65 (manufactured by Nikko Chemicals Co., Ltd.); Stearyl Alcohol NX (manufactured by KOKYU ALCOHOL KOGYO CO., LTD.); NIKKOL Batyl Alcohol EX (manufactured by Nikko Chemicals Co., Ltd.); and the like.

### (D) Silicone Oil

The silicone oil used in the present invention has a kinematic viscosity at 25° C. of 100 cSt or less. When the kinematic viscosity exceeds 100 cSt, dispersion state of the partially-crosslinked or crosslinked dimethylpolysiloxane may deteriorate, and thus it is not preferred.

When the silicone oil (D) of the present invention is used in the oil-in-water emulsion cosmetic, it is preferably 5 to 30% by mass in the cosmetic. When it exceeds 30%, emulsification may be unsuccessful. When it is less than 5%, aggregation of the partially-crosslinked or crosslinked dimethylpolysiloxane may occur.

The silicone oil used in the present invention is not limited in particular; however, the silicone oil preferably has a phenyl group or a methyl group.

Among the above, it is preferred that the silicone oil has a phenyl group in terms of compatibility with solid oils.

Examples of commercially available products of the silicone oil (D) include: Silicone KF-56 (manufactured by Shin-Etsu Chemical Co., Ltd.); KF-96A-6T (manufactured by Shin-Etsu Chemical Co., Ltd.); and the like.

The oil-in-water emulsion cosmetic according to the present invention needs to satisfy the following conditions (1) to (3).

### Condition (1)

(A)/((B)+(C)+(D))=0.01 to 1
The weight ratio of A/(B+C+D) represents a range capable of stably emulsifying the components (A) to (D). Relative to the blending amount of the component (A), the blending amount of the components (B) to (D) needs to be within the range of (A)/((B)+(C)+(D))=0.01 to 1, preferably 0.01 to 0.5, and particularly preferably 0.01 to 0.2. When (A)/((B)+(C)+(D)) is less than 0.01, emulsification may be unsuccessful or aggregation of the partially-crosslinked or crosslinked dimethylpolysiloxane may occur, and thus it is not preferred. When (A)/((B)+(C)+(D)) exceeds 1, sliminess during application and stickiness after application may become stronger, and thus it is not preferred.

### Condition (2)

(A)/(C)=0.01 to 1
The contents of the components (A) and (C) need to be within the range of (A)/(C)=0.01 to 1 in weight ratio, preferably 0.1 to 1, and particularly preferably 0.1 to 0.5. When (A)/(C) is less than 0.01, emulsification may be unsuccessful or aggregation of the partially-crosslinked or crosslinked dimethylpolysiloxane may occur, and thus it is not preferred. When (A)/(C) exceeds 1, sliminess during application and stickiness after application may become stronger, and thus it is not preferred.

### Condition (3)

The oil-in-water emulsion cosmetic has a viscosity at 25° C. of 10 to 100000 mPa·s, and more preferably 1000 to 50000. When it is less than 10 mPa·s, aggregation of the partially-crosslinked or crosslinked dimethylpolysiloxane may occur during long-term storage, and thus it is not preferred. When it exceeds 100000 mPa·s, smooth and good texture upon use may not be achieved, and thus it is not preferred.

The oil-in-water emulsion cosmetic according to the present invention preferably has an emulsion particle size of 100 μm or less. When it exceeds 100 μm, aggregation of the partially-crosslinked or crosslinked dimethylpolysiloxane may occur, and thus it is not preferred.

In the present invention, the silicone surfactant (E) can be blended.

When the silicone surfactant (E) it blended to the oil-in-water emulsion cosmetic according to the present invention, it is preferably 0.05 to 2.0% by mass in the cosmetic because dispersion of the partially-crosslinked or crosslinked dimethylpolysiloxane may be improved and coalescence of the partially-crosslinked or crosslinked dimethylpolysiloxane during long-term storage may be suppressed. When it is less than 0.05% by mass, it is ineffective in dispersion of the partially-crosslinked or crosslinked dimethylpolysiloxane and suppression of coalescence, and thus not preferred. When it exceeds 2.0% by mass, stickiness after application may become stronger, and thus it is not preferred.

The silicone surfactant (E) used in the present invention is not limited in particular; however, polyglyceryl-modified silicone, polyether-modified silicone, and polyether-modified/alkyl-modified silicone are preferred. Among the above, in particular, the silicone surfactant preferably has a silicone chain (siloxane chain) as a main chain and a hydrophilic group having a polyether group as a side chain. The silicone surfactant preferably has a pendant-shaped (comb-shaped) structure in which a plurality of polyoxyalkelene groups (polyether chain) is introduced to the silicone chain as the side chains. If the silicone surfactant has the pendant-shaped structure, dispersion stability of the partially-crosslinked or crosslinked dimethylpolysiloxane can be enhanced.

In terms of suppression of coalescence of the partially-crosslinked or crosslinked dimethylpolysiloxane, polyether (3-40) modified polysiloxane is preferred among the above. PEG-10 dimethicone is particularly preferred.

Moreover, HLB is preferably less than 10 in terms of improvement in dispersion and suppression of aggregation of the partially-crosslinked or crosslinked dimethylpolysiloxane.

Examples of commercially available products of the silicone surfactant (E) include: KF-6017P (PEG-10 dimethicone, manufactured by Shin-Etsu Silicones); KF-6048 (cetyl PEG/PPG-10/1 dimethicone, manufactured by Shin-Etsu Silicones); and Silicone KF-6109 (bis-butyldimethicone polyglyceryl-3, manufactured by Shin-Etsu Silicones).

In the present invention, the water-soluble thickener (F) can be blended.

As the water-soluble thickener, for example, taurate-based synthetic polymer and/or acrylate-based synthetic polymer can be used.

For example, as the taurate-based synthetic polymer type thickener, a polymer and/or a copolymer (including a crosslinked polymer) having 2-acrylamido-2-propanesulphonic acid (acryloyl dimethyl tauric acid) or a salt thereof (AMPS structure) as a constituent unit may be used. As such thickener, for example, at least one selected from Carboxyvinyl polymer (Carbopol 981, manufactured by Lubrizol Corporation), Ammonium Acryloyldimethyltaurate/Beheneth-25 Methacrylate Crosspolymer (Aristoflex® HMB, Clariant (Japan) K.K.), Ammonium Acryloyldimethyltaurate/VP Copolymer (Aristoflex® AVC, Clariant (Japan) K.K.), Ammonium Acryloyldimethyltaurate/Carboxyethyl Acrylate Crosspolymer (Aristoflex® TAC, Clariant (Japan) K.K.), Polyacrylate Crosspolymer-11 (Aristoflex® Velvet, Clariant (Japan) K.K.), Dimethylacrylamide/Sodium Acryloyldimethyl 16 Taurate Crosspolymer (SU-GEL, TOHO Chemical Co., Ltd.), Hydroxyethyl Acrylate/Sodium Acryloyldimethyltaurate Copolymer (SEPINOV EMT10 PINOV, SEPPIC Inc.), Sodium Acrylate/Acryloyldimethyltaurate/Dimethylacrylamide Crosspolymer (SEPINOV P88, SEPPIC Inc.), Sodium Acrylate/Sodium Acryloyldimethyltaurate Copolymer (SIMUGEL EG, SEPPIC Inc.), Sodium Acryloyldimethyltaurate/Methacrylamidolauric Acid Copolymer (AMO-51, DAITO KASEI KOGYO CO., LTD.) and Aacrylamide/Sodium Acryloyldimethyltaurate/Acrylic Acid Copolymer (ACUDYNE SCP, Dow Chemical Company) can be used.

For example, as the acrylate-based synthetic polymer type thickener, Acrylates/Steareth-20 Methacrylate Copolymer (ACULYN® 22, Dow Chemical Company) and Acrylates/C10-30 Alkyl Acrylate Crosspolymer (PEMULEN® TR-2) can be used.

The content of the water-soluble thickener in the composition of the present disclosure relative to the mass of the composition is preferably 0.05% by mass or greater, and more preferably 0.08% by mass or greater. When the content of the water-soluble thickener is less than 0.05% by mass, emulsion stability deteriorates, and dispersibility of the partially-crosslinked or crosslinked dimethylpolysiloxane deteriorates, too. The content of the water-soluble thickener relative to the mass of the composition is preferably 2% by mass or less, and more preferably 1.5% by mass or less. When the content of the water-soluble thickener exceeds 2% by mass, feeling in use required by the composition of the present disclosure may be deteriorated.

Other thickeners can be added within the range of not inhibiting the effect of the present invention. Examples of other thickeners may include gum arabic, carrageenan, karaya gum, tragacanth gum, carob gum, quince seed (marmelo), casein, dextrin, gelatin, sodium pectate, sodium alginate, methylcellulose, ethyl cellulose, carboxymethyl cellulose (CMC), hydroxyethyl cellulose, hydroxypropyl cellulose, polyvinyl alcohol (PVA), polyvinyl methyl ether (PVM), PVP (polyvinyl pyrrolidone), sodium polyacrylate, locustbean gum, guar gum, tamarind gum, dialkyldimethylammonium sulfate cellulose, xanthan gum, magnesium aluminum silicate, bentonite, hectorite, magnesium aluminum silicate (veegum), laponite, silicic anhydride and the like.

In the present invention, when an animal/plant-derived or chemically synthesized oily component having an IOB value in an organic conceptual diagram (Qualitative analysis of organic substance: by Makoto Fujita) is 0 to 0.2 is blended separately from the component (C), it is preferred in terms of improvement in solubility of solid oil components. When it exceeds 0.2, solid oil components may not dissolve, and thus it is not preferred.

The animal/plant-derived or chemically synthesized oily component having an IOB value of 0 to 0.2 is not limited in particular; however, an ester oil that is liquid at 25° C. is preferred in terms of improvement in solubility of solid oil components.

The animal/plant-derived or chemically synthesized oily component having an IOB value of 0 to 0.2 is preferably 1 to 20% by mass in the cosmetic. When it is less than 1% by mass, solid oil components may not dissolve sufficiently, and thus it is not preferred. When it exceeds 20% by mass, aggregation of the partially-crosslinked or crosslinked dimethylpolysiloxane may occur, and thus it is not preferred.

A nonionic surfactant can be blended to the present invention. When the nonionic surfactant is blended, it is preferably 0.01 to 1% by mass in the cosmetic because emulsification property may be improved. When it is less than 0.01% by mass, it is not preferred because emulsification property may not change. When it exceeds 1% by mass, sliminess during application and stickiness after application may become stronger, and thus it is not preferred.

Examples of the nonionic surfactant include polyoxyethylene hydrogenated castor oil, glycerin fatty acid ester, polyoxyethylene fatty acid sorbitan, and the like.

Among the above, alkyl monoglyceride and dialkyl monoglyceride having CCP (average critical packing parameter) of 0.8 to 1.2, and a sorbitan mono fatty acid ester (C10 to C24) are preferred.

Average critical packing parameter in the present invention “Packing parameter” is a value expressed by V/(1·a) when the volume occupied by the hydrophobic group in the micelle shell is V, the length of the hydrophobic group in the micelle shell is 1, and the area occupied by the hydrophilic group in the boundary between micelle-solution is a; and it is a parameter for explaining the relationship between the molecular structure and the association structure. The relationship between the critical packing parameter and the association structure that can be taken stably is indicated by Israelachvili (“Intermolecular and Surface Forces, with Applications to Colloidal and Biological Systems”, Academic Press, London, 1985, p 247; “Intermolecular and Surface Forces”, Second edition, J. N. Israelachvili, translated by KONDO Tamotsu, OSHIMA Hiroyuki, Asakura Shoten, 1996, p 369; “Surfactants and Interfacial Phenomena”, M. J. Rosen, supervised by TSUBONE Kazuyuki, SAKAMOTO Kazutami, Fragrance Journal Ltd., 1995, p 116).

In the present invention, an amphoteric surfactant can be blended. When the amphoteric surfactant is blended, it is preferably 0.01 to 1% by mass in the cosmetic because dispersibility of the partially-crosslinked or crosslinked dimethylpolysiloxane improves. When it is less than 0.01% by mass, dispersibility of the partially-crosslinked or crosslinked dimethylpolysiloxane may not be affected, and thus it is not preferred. When it exceeds 1% by mass, stickiness may be felt after application, and thus it is not preferred.

Examples of the amphoteric surfactant include lauryl dimethylaminoacetic acid betaine, coconut oil fatty acid acylglycine sodium, and the like.

Among the above, lauryl dimethylaminoacetic acid betaine is preferred.

In the present invention, polyols and sugar alcohols can be comprised.

Examples of the polyols and sugar alcohols include glycerin, butylene glycol, erythritol, and the like. In particular, a value of water/octanol partition coefficient (log P value) that shows water-solubility and fat-solubility is preferably within a range of −3.0 to −1.0.

The blending amount of the polyols and sugar alcohols is preferably 1 to 30% by mass in the cosmetic. When it is less than 1% by mass, moisturizing effect sufficient as a skin care may not be achieved, and thus it is not preferred. When it exceeds 30% by mass, sliminess during application and stickiness after application may become stronger, and thus it is not preferred.

In the present invention, a low-soluble drug can be comprised.

Examples of the low-soluble drug include Coenzyme Q10, stearyl glycyrrhetinate, astaxanthin, tranexamic acid, allantoin, alkoxy salicylic acid, a whitening agent (other components), and the like. In particular, the value of water/octanol partition coefficient (log P value) that shows water-solubility and fat-solubility is preferably within a range of 10 to 30.

### [Other Components]

The cosmetic according to the present invention may comprise, as appropriate and as necessary, other components such as moisturizers, water-soluble polymers, thickeners, film-forming agents, ultraviolet light absorbers, sequestrants, lower alcohols, polyhydric alcohols, saccharides, amino acids, organic amines, polymeric emulsions, pH adjusters, skin nutrients, vitamins, antioxidants, antioxidant aids, perfumes, and water in amounts that do not inhibit the effects of the present invention, and it can be manufactured by a common method in accordance with the dosage form of target.

Examples of the moisturizers include polyethylene glycol, propylene glycol, glycerin, 1,3-butylene glycol, xylitol, sorbitol, maltitol, chondroitin sulfate, hyaluronic acid, mucoitin sulfate, charonic acid, atelocollagen, cholesteryl-12-hydroxystearate, sodium lactate, bile salt, dl-pyrrolidone carboxylate, alkyleneoxide derivative, short-chain soluble collagen, diglycerin (EO)PO adduct, chestnut rose extract, yarrow extract, melilot extract, and the like.

Examples of the natural water-soluble polymer include: plant-based polymers (such as gum arabic, gum tragacanth, galactan, guar gum, locust bean gum, gum karaya, carrageenan, pectine, agar, quince seed (marmelo), algae colloid (brown algae extract), starch (rice, corn, potato, wheat), and glicyrrhizic acid); microorganism-based polymers (such as xanthan gum, dextran, succinoglycan, pullulan, etc); animal-based polymers (such as collagen, casein, albumin, gelatine, etc); and the like.

Examples of the semisynthetic water-soluble polymer include: starch-based polymers (such as carboxymethyl starch, methylhydroxypropyl starch, etc); cellulose-based polymers (such as methylcellulose, ethylcellulose, methylhydroxypropylcellulose, hydroxyethylcellulose, cellulose sodium sulfate, hydroxypropylcellulose, carboxymethylcellulose, sodium calboxymethyl cellulose, crystalline cellulose, cellulose powder, etc); algin acid-based polymers (such as sodium alginate, propylene glycol alginate ester, etc); and the like.

Examples of the synthetic water-soluble polymer include vinyl-based polymers (such as polyvinyl alcohol, polyvinyl methyl ether, polyvinylpyrrolidone, carboxyvinylpolymer, etc); polyoxyethylene-based polymers (such as polyoxyethylenepolyoxypropylene copolymer such as polyethylene glycol 20,000, 40,000 and 60,000, etc); acrylic polymers (such as sodium polyacrylate, polyethylacrylate, polyacrylamide, etc); polyethyleneimine; cationic polymers; and the like.

Examples of thickeners include gum arabic, carrageenan, karaya gum, tragacanth gum, carob gum, quince seed (marmelo), casein, dextrin, gelatin, sodium pectate, sodium alginate, methyl cellulose, ethyl cellulose, CMC, hydroxyethyl cellulose, hydroxypropyl cellulose, PVA, PVM, PVP, sodium polyacrylate, carboxyvinyl polymer, locust bean gum, guar gum, tamarind gum, dialkyldimethylammonium sulfate cellulose, xanthan gum, aluminum magnesium silicate, bentonite, hectorite, aluminum magnesium silicate (veegum), laponite, silicic acid anhydride, and the like.

Examples of the ultraviolet light absorbers include benzoic acid family ultraviolet light absorbers (such as p-aminobenzoic acid (hereinafter abbreviated as PABA), PABA monoglycerine ester, N,N-dipropoxy PABA ethyl ester, N,N-diethoxy PABA ethyl ester, N,N-dimethyl PABA ethyl ester, N,N-dimethyl PABA butyl ester, N,N-dimethyl PABA ethyl ester, etc); anthranilic acid family ultraviolet light absorbers (such as homomenthyl N-acetylanthranilate etc); salicylic acid family ultraviolet light absorbers (such as amyl salicylate, menthyl salicylate, homomenthyl salicylate, octyl salicylate, phenyl salicylate, benzyl salicylate, p-isopropanolphenyl salicylate, etc); cinnamic acid family ultraviolet light absorbers (such as octyl methoxycinnamate, ethyl-4-isopropylcinnamate, methyl-2,5-diisopropylcinnamate, ethyl-2,4-diisopropylcinnamate, methyl-2,4-diisopropylcinnamate, propyl-p-methoxycinnamate, isopropyl-p-methoxycinnamate, isoamyl-p-methoxycinnamate, octyl-p-methoxycinnamate (2-ethylhexyl-p-methoxycinnamate), 2-ethoxyethyl-p-methoxycinnamate, cyclohexyl-p-methoxycinnamate, ethyl-α-cyano-β-phenylcinnamate, 2-ethylhexyl-α-cyano-β-phenylcinnamate, glyceryl mono-2-ethylhexanoyl-diparamethoxy cinnamate, etc); benzophenone family ultraviolet light absorbers (such as 2,4-dihydroxybenzophenone, 2,2′-dihydroxy-4-methoxybenzophenone, 2,2′-dihydroxy-4,4′-dimethoxybenzophenone, 2,2′,4,4′-tetrahydroxybenzophenone, 2-hydroxy-4-methoxybenzophenone, 2-hydroxy-4-methoxy-4′-methylbenzophenone, 2-hydroxy-4-methoxybenzophenone-5-sulfonate, 4-phenylbenzophenone, 2-ethylhexyl-4′-phenyl-benzophenone-2-carboxylate, 2-hydroxy-4-n-octoxybenzophenone, 4-hydroxy-3-carboxybenzophenone, etc); 3-(4′-methylbenzylidene)-d,l-camphor, 3-benzylidene-d,l-camphor; 2-phenyl-5-methylbenzoxazol; 2,2′-hydroxy-5-methylphenylbenzotriazol, 2-(2′-hydroxy-5′-t-octylphenyl) benzotriazol; 2-(2′-hydroxy-5′-methylphenylbenzotriazol; dibenzalazine; dianisoylmethane; 4-methoxy-4′-t-butyldibenzoylmethane; 5-(3,3-dimethyl-2-norbornylidene)-3-pentane-2-one, dimorpholinopyridazinone; 2-ethylhexyl-2-cyano-3,3-diphenylacrylate; 2,4-bis-{[4-(2-ethylhexyloxy)-2-hydroxy]-phenyl}-6-(4-methoxyphenyl)-(1,3,5)-triazine; and the like.

Examples of the sequestrant include 1-hydroxyethane-1,1-diphosphonic acid, 1-hydroxyethane-1,1-diphosphonic acid 4Na salt, disodium edetate, trisodium edetate, tetrasodium edetate, sodium citrate, sodium polyphosphate, sodium metaphosphate, gluconic acid, phosphoric acid, citric acid, ascorbic acid, succinic acid, edetic acid, trisodium hydroxyethyl ethylenediamine triacetate, and the like.

Examples of the lower alcohol include ethanol, propanol, isopropanol, isobutyl alcohol, t-butyl alcohol, and the like.

Examples of the polyhydric alcohol include dihydric alcohols (such as ethylene glycol, propylene glycol, trimethylene glycol, 1,2-butylene glycol, 1,3-butylene glycol, tetramethylene glycol, 2,3-butylene glycol, pentamethylene glycol, 2-butene-1,4-diol, hexylene glycol, octylene glycol, etc); trihydric alcohols (such as glycerin, trimethylolpropane, etc); tetrahydric alcohols (such as pentaerythritol such as 1,2,6-hexanetriol, etc); pentahydric alcohols (such as xylitol, etc); hexahydric alcohols (such as sorbitol, mannitol, etc); polyhydric alcohol polymers (such as diethylene glycol, dipropylene glycol, triethylene glycol, polypropylene glycol, tetraethylene glycol, diglycerin, polyethylene glycol, triglycerin, tetraglycerin, polyglycerin, etc); dihydric alcohol alkyl ethers (such as ethylene glycol monomethyl ether, ethylene glycol monoethyl ether, ethylene glycol monobutyl ether, ethylene glycol monomphenyl ether, ethylene glycol monohexyl ether, ethylene glycol mono2-methylhexyl ether, ethylene glycol isoamyl ether, ethylene glycol benzil ether, ethylene glycol isopropyl ether, ethylene glycol dimethyl ether, ethylene glycol diethyl ether, ethylene glycol dibutyl ether, etc); dihydric alcohol alkyl ethers (such as diethylene glycol monomethyl ether, diethylene glycol monoethyl ether, diethylene glycol monombutyl ether, diethylene glycol dimethyl ether, diethylene glycol diethyl ether, diethylene glycol butyl ether, diethylene glycol methylethyl ether, triethylene glycol monomethyl ether, triethylene glycol monoethyl ether, propylene glycol monomethyl ether, propylene glycol monoethyl ether, propylene glycol monobutyl ether, propylene glycol isopropyl ether, dipropylene glycol methyl ether, dipropylene glycol ethyl ether, dipropylene glycol butyl ether, etc); dihydric alcohol ether ethers (such as ethylene glycol monomethyl ether acetate, ethylene glycol monoethyl ether acetate, ethylene glycol monobutyl ether acetate, ethylene glycol monophenyl ether acetate, ethylene glycol diadipate, ethylene glycol disaccinate, diethylene glycol mono ethyl ether acetate, diethylene glycol monobutyl ether acetate, propylene glycol monomethyl ether acetate, propylene glycol monoethyl ether acetate, propylene glycol monopropyl ether acetate, propylene glycol monophenyl ether acetate, etc); glycerin monoalkyl ethers (such as xyl alcohol, selachyl alcohol, batyl alcohol, etc); sugar alcohols (such as sorbitol, maltitol, maltotriose, mannitol, sucrose, erythritol, glucose, fructose, starch sugar, maltose, xylitose, starch sugar hydrogenated alcohol, etc); glysolide; tetrahydrofurfuryl alcohol; POE-tetrahydrofurfuryl alcohol; POP-butyl ether; POP/POE-butyl ether; tripolyoxypropylene glycerin ether; POP-glycerin ether; POP-glycerin ether phosphoric acid; POP/POE-pentaerythritol ether; polyglycerin; and the like.

Examples of the monosaccharides include triose (such as D-glyceryl aldehyde, dihydroxyacetone, etc); tetrose (such as D-erythrose, D-erythrulose, D-threose, erythritol, etc); pentaose (such as L-arabinose, D-xylose, L-lyxose, D-arabinose, D-ribose, D-ribulose, D-xylulose, L-xylulose, etc); hexalose (such as D-glucose, D-talose, D-psicose, D-galactose, D-fructose, L-galactose, L-mannose, D-tagatose, etc); heptose (such as aldoheptose, heptulose, etc); octose (such as octulose, etc); deoxy sugar (such as 2-deoxy-D-ribose, 6-deoxy-L-galactose, 6-deoxy-L-mannose, etc); amino sugar (such as D-glucosamine, D-galactosamine, sialic acid, amino uronic acid, muramic acid, etc); uronic acid (such as D-grucuronic acid, D-mannuronic acid, L-guluronic acid, D-garacturonic acid, L-iduronic acid, etc) and the like.

Examples of the oligosaccharide include sucrose, guntianose, umbelliferose, lactose, planteose, isolignoses, α,α-trehalose, raffinose, lignoses, umbilicin, stachyose, verbascoses, and the like.

Examples of the polysaccharide include cellulose, quince seed, chondroitin sulfate, starch, galactan, dermatan sulfate, glycogen, gum arabic, heparansulfate, hyaluronic acid, gum tragacanth, keratan sulfate, chondoroitin, xanthan gum, mucoitin sulfate, guar gum, dextran, keratosulfate, locust bean gum, succinoglycan, caronic acid, and the like.

Examples of the amino acid include neutral amino acids (such as threonine, cysteine, etc); basic amino acids (such as hydroxylysine, etc) and the like. Examples of the amino acid derivative include sodium acyl sarcosinate (sodium lauroyl sarcosinate), acyl glutamate, sodium acyl β-alanine, glutathione, pyrrolidone carboxylate, and the like.

Examples of the organic amine include monoethanolamine, diethanolamine, triethanolamine, morpholine, triisopropanolamine, 2-amino-2-methyl-1,3-propanediol, 2-amino-2-methyl-1-propanol, and the like.

Examples of the polymer emulsion include acrylic resin emulsion, ethyl polyacrylate emulsion, solution of acrylic resin, polyacrylalkylester emulsion, polyvinyl acetate resin emulsion, natural rubber latex, and the like.

Examples of the pH modifier include buffers such as lactic acid-sodium lactate, citric acid-sodium citrate, succinic acid-sodium succinate, and the like.

Examples of the vitamins include vitamin A, B1, B2, B6, C, E and derivatives thereof, pantothenic acid and derivatives thereof, biotin, and the like.

Examples of the anti-oxidant include tocopherols, dibutyl hydroxy toluene, butyl hydroxy anisole, gallic acid esters, and the like.

Examples of the anti-oxidant aid include phosphoric acid, citric acid, ascorbic acid, maleic acid, malonic acid, succinic acid, fumaric acid, cephalin, hexamethaphosphate, phytic acid, ethylenediaminetetraacetic acid, and the like.

Examples of other blendable components include antiseptic agents (such as ethylparaben, butylparaben, chlorphenesin, phenoxyethanol, etc); antiphlogistics (such as glycyrrhizinic acid derivatives, glycyrrhetic acid derivatives, salicylic acid derivatives, hinokitiol, zinc oxide, allantoin, etc); skin-whitening agents (such as placental extract, saxifrage extract, arbutin, etc); various extracts (such as phellodendron bark (cork tree bark), coptis rhizome, lithospermum, peony, swertia herb, birch, sage, loquat, carrot, aloe, mallow, iris, grape, coix seed, sponge gourd, lily, saffron, cnidium rhizome, ginger, hypericum, restharrow, garlic, red pepper, citrus unshiu, Japanese angelica, seaweed, etc); activators (such as royal jelly, photosenstizer, cholesterol derivatives, etc); blood circulation promoters (such as nonylic acid vanillylamide, nicotine acid benzyl ester, nicotine acid β-butoxyethyl ester, capsaicin, zingerone, cantharides tincture, ichthammol, tannic acid, α-borneol, tocopheryl nicotinate, meso-inositol hexanicotinate, cyclandelate, cinnarizine, tolazoline, acetylcholine, verapamil, cepharanthine, γ-oryzanol, etc); antiseborrheric agents (such as sulfur, thianthl, etc); anti-inflammatory agents (such as tranexamic acid, thiotaurine, hypotaurine, etc); and the like.

In addition, sequestrants such as disodium edetate, trisodium edetate, sodium citrate, sodium polyphosphate, sodium metaphosphate, gluconic acid, malic acids and the like; caffeine, tannin, verapamil, tranexamic acid and derivatives thereof; various crude drug extracts such as licorice, Chinese quince, Pyrola japonica and the like; drugs such as tocopherol acetate, glycyrrhetinic acid, glycyrrhizic acid and derivatives thereof, or salts thereof; skin-whitening agents such as vitamin C, magnesium ascorbyl phosphate, ascorbic acid glucoside, arbutin, kojic acid and the like; amino acids such as arginine and lysine and the like and derivatives thereof; and saccharides such as fructose, mannose, erythritol, trehalose, xylitol and the like further may be blended as necessary.

The oil-in-water emulsion cosmetic according to the present invention can be in any product form. Specifically, it can be skincare cosmetics such as moisturizing gels, massage gels, serums, cosmetic lotions, and emulsions; makeup cosmetics; sun-care products; hair cosmetics such as hair-setting agents and hair gels; hair dyes; and the like.

### [Manufacturing Method of the Oil-in-Water Emulsion Cosmetic According to the Present Invention]

It can be manufactured by a well-known method. For example, an anionic surfactant is dissolved to water (A). Oil is dissolved at high temperature (B). A and B are mixed to give an oil-in-water emulsion cosmetic of target in a suitable viscosity region.

### Examples

The present invention is described with reference to examples below. However, the present invention is not limited to the following examples. The blending amounts of the components are in “% by mass” relative to the system to which the component is blended, unless otherwise specified.

Before describing the examples, evaluation methods of the oil-in-water emulsion cosmetic according to the present invention are described.

### Evaluation (1): Evaluation of Low-Speed Stirring

In Low-speed stirring, 200 g of a sample was stirred by a three-one motor at 40 rpm for four hours to evaluate a state of aggregation of the partially-crosslinked or crosslinked dimethylpolysiloxane and a change in appearance.

### <Evaluation Criteria>

A: Aggregation of the partially-crosslinked or crosslinked dimethylpolysiloxane was not observed.
B: Aggregation of the partially-crosslinked or crosslinked dimethylpolysiloxane was hardly observed.
C: Aggregation of the partially-crosslinked or crosslinked dimethylpolysiloxane was observed.

### Evaluation (2): Evaluation of Rolling Test (4 hr)

In Rolling test, 30 ml of the composition was put into a 50 ml screw tube, and the screw tube was rotated at a rotation speed of 45 rpm at 25° C. for 4 hours to evaluate a state of aggregation of the partially-crosslinked or crosslinked dimethylpolysiloxane and a change in appearance.

### <Evaluation Criteria>

A: Aggregation of the partially-crosslinked or crosslinked dimethylpolysiloxane was not observed.
B: Aggregation of the partially-crosslinked or crosslinked dimethylpolysiloxane was hardly observed.
C: Aggregation of the partially-crosslinked or crosslinked dimethylpolysiloxane was observed.

TABLE 1

| | Labelling Name of | | Test | Test | Test | Test | Test |
|---|---|---|---|---|---|---|---|
| Classification | Cosmetic Ingredient | | Example 1 | Example 2 | Example 3 | Example 4 | Example 5 |
| Activator | Sodium methyl | A | 0.2 | 0.4 | 0.2 | 0.2 | 0.2 |
| | stearoyl taurate | | | | | | |
| Partially-crosslinked | Polysilicone-11 (*3) | B | 0.6 | 0.6 | 0.6 | 0.6 | 0.6 |
| or crosslinked | Alkyl-crosslinked | B | — | — | — | — | — |
| dimethylpolysiloxane | polydimethylsiloxane | | | | | | |
| | (*4) | | | | | | |
| | Dimethicone/phenyl | B | — | — | — | — | — |
| | vinyl dimethicone | | | | | | |
| | crosspolymer (*5) | | | | | | |
| Oily component | Stearyl alcohol | C | 0.36 | 0.3 | 0.36 | 0.36 | 0.36 |
| | Behenyl alcohol | C | 1.32 | 1.2 | 1.32 | 1.32 | 1.32 |
| | Diphenylsiloxy | D | 3 | 3 | — | — | — |
| | phenyl trimethicone | | | | | | |
| | Dimethicone | D | 8.4 | 8.4 | 10.4 | 10.4 | 10.4 |
| | Pentaerythritol | | 2 | 2 | 3 | 3 | 3 |
| | tetra(2-ethylhexanoate) | | | | | | |
| Activator | PEG-10 dimethicone | E | 0.4 | 0.4 | 0.4 | — | — |
| | Bis-butyldimethicone | E | — | — | — | 0.4 | — |
| | polyglyceryl-3 (*1) | | | | | | |
| | Bis(PEG/PPG-20/ | E | — | — | — | — | 0.4 |
| | 5)PEG/PPG-20/5 | | | | | | |
| | dimethicone, Methoxy | | | | | | |
| | PEG/PPG-25/4 | | | | | | |
| | dimethicone, | | | | | | |
| | Caprylic/capric | | | | | | |
| | triglyceride (*2) | | | | | | |
| Thickener | Carboxyvinyl polymer | F | 0.14 | 0.14 | 0.14 | 0.14 | 0.14 |
| | (*4) | | | | | | |
| Water | Water | | to 100 | to 100 | to 100 | to 100 | to 100 |
| Alcohol | Ethanol | | 2 | 2 | 2 | 2 | 2 |
| Moisturizer | Concentrated glycerin | | 4 | 4 | 4 | 4 | 4 |
| | Dipropylene glycol | | 7 | 7 | 7 | 7 | 7 |
| Neutralizer | Potassium hydroxide | | 0.1 | 0.1 | 0.1 | 0.1 | 0.1 |
| Chelating agent | Disodium edetate | | Suitable | Suitable | Suitable | Suitable | Suitable |
| | | | amount | amount | amount | amount | amount |
| Preservative | Methyl | | Suitable | Suitable | Suitable | Suitable | Suitable |
| | paraoxybenzoate | | amount | amount | amount | amount | amount |
| | Phenoxyethanol | | Suitable | Suitable | Suitable | Suitable | Suitable |
| | | | amount | amount | amount | amount | amount |
| | A/(B + C + D) | | 0.015 | 0.03 | 0.016 | 0.016 | 0.016 |
| | A/C | | 0.119 | 0.267 | 0.119 | 0.119 | 0.119 |

| | | | | | |
|---|---|---|---|---|---|
| Viscosity | 6760 | 6550 | 8330 | 6000 | 6690 |
| Low-speed stirring (40 rpm, 4 hr, 200 g) | A | A | A | B | B |
| Rolling test (4 hr) | A | A | A | B | B |

TABLE 2

| | Labelling Name of | | Test | Test | Test | Test | Test | Test |
|---|---|---|---|---|---|---|---|---|
| Classification | Cosmetic Ingredient | | Example 6 | Example 7 | Example 8 | Example 9 | Example 10 | Example 11 |
| Activator | Sodium methyl | A | 0.2 | 0.2 | 0.2 | 0.2 | 1.5 | 0.2 |
| | stearoyl taurate | | | | | | | |
| Partially-crosslinked | Polysilicone-11 (*3) | B | 0.6 | — | — | 0.6 | 0.6 | 0.6 |
| or crosslinked | Alkyl-crosslinked | B | — | 0.8 | — | — | — | — |
| dimethylpolysiloxane | polydimethylsiloxane | | | | | | | |
| | (*4) | | | | | | | |
| | Dimethicone/phenyl | B | — | — | 0.8 | — | — | — |
| | vinyl dimethicone | | | | | | | |
| | crosspolymer (*5) | | | | | | | |
| Oily component | Stearyl alcohol | C | 0.36 | 0.36 | 0.36 | 0.36 | 0.36 | 0.36 |
| | Behenyl alcohol | C | 1.32 | 1.32 | 1.32 | 1.32 | 1.32 | 1.32 |
| | Diphenylsiloxy | D | — | — | 4.2 | — | — | — |
| | phenyl trimethicone | | | | | | | |
| | Dimethicone | D | 10.4 | 10.2 | 6 | 10.4 | 10.4 | 5.4 |
| | Pentaerythritol | | 3 | 3 | 3 | 3 | 3 | 3 |
| | tetra(2-ethylhexanoate) | | | | | | | |
| Activator | PEG-10 dimethicone | E | 0.4 | 0.4 | 0.4 | — | 0.4 | 0.4 |
| | Bis-butyldimethicone | E | — | — | — | — | — | — |
| | polyglyceryl-3 (*1) | | | | | | | |
| | Bis(PEG/PPG-20/ | E | — | — | — | — | — | — |
| | 5)PEG/PPG-20/5 | | | | | | | |
| | dimethicone, Methoxy | | | | | | | |
| | PEG/PPG-25/4 | | | | | | | |
| | dimethicone, | | | | | | | |
| | Caprylic/capric | | | | | | | |
| | triglyceride (*2) | | | | | | | |
| Thickener | Carboxyvinyl polymer | F | 0.14 | 0.14 | 0.14 | 0.14 | 0.14 | 0.14 |
| Water | Water | | to 100 | to 100 | to 100 | to 100 | to 100 | to 100 |
| Alcohol | Ethanol | | 2 | 2 | 2 | 2 | 2 | 2 |
| Moisturizer | Concentrated glycerin | | 4 | 4 | 4 | 4 | 4 | 4 |
| | Dipropylene glycol | | 7 | 7 | 7 | 7 | 7 | 7 |
| Neutralizer | Potassium hydroxide | | 0.1 | 0.1 | 0.1 | 0.1 | 0.1 | 0.1 |
| Chelating agent | Disodium edetate | | Suitable | Suitable | Suitable | Suitable | Suitable | Suitable |
| | | | amount | amount | amount | amount | amount | amount |
| Preservative | Methyl | | Suitable | Suitable | Suitable | Suitable | Suitable | Suitable |
| | paraoxybenzoate | | amount | amount | amount | amount | amount | amount |
| | Phenoxyethanol | | Suitable | Suitable | Suitable | Suitable | Suitable | Suitable |
| | | | amount | amount | amount | amount | amount | amount |
| | A/(B + C + D) | | 0.016 | 0.016 | 0.016 | 0.016 | 0.118 | 0.026 |
| | A/C | | 0.119 | 0.119 | 0.119 | 0.119 | 0.893 | 0.119 |

| | | | | | | |
|---|---|---|---|---|---|---|
| Viscosity | 8690 | 9450 | 9240 | 8140 | 4410 | 6280 |
| Low-speed stirring (40 rpm, 4 hr, 200 g) | A | A | A | C | A | B |
| Rolling test (4 hr) | B | A | A | C | B | B |

(*1) SILICONE KF-6109 (manufactured by Shin-Etsu Chemical Co., Ltd.)
(*2) ABIL Care XL 80 (manufactured by Degussa Japan Co., Ltd.)
(*3) Gransil DMCM-5 (manufactured by Grant Industries, Inc.)
(*4) DOW CORNING® 9041 SILICONE ELASTOMER BLEND (manufactured by Dow Corning Toray CO., Ltd.)
(*5) KSG-18A (manufactured by Shin-Etsu Chemical Co., Ltd.)

From the above test examples, it was found that aggregation of the partially-crosslinked or crosslinked dimethylpolysiloxane was observed when the silicone surfactant (E) was not blended.

1. An oil-in-water emulsion cosmetic comprising the following components (A) to (D) and satisfying the following conditions (1) to (3):
(A) an anionic surfactant represented by the following general formula and has a sulfonic acid group in a molecule.
R₁CO-a-(CH₂)nSO₃M₁ (I)
in the formula (I): R₁CO represents a saturated or unsaturated fatty acid residue (acyl group) having 10 to 22 carbon atoms on average; a represents —O— or —NR₂ (R₂ represents a hydrogen atom or a C₁₋₃ alkyl group); M₁ represents a hydrogen atom, alkaline metal, alkaline earth metal, ammonium, or organic amine; and n represents an integer of 1 to 3,
(B) a partially-crosslinked or crosslinked dimethylpolysiloxane
(C) an animal/plant-derived or chemically synthesized oily component that is solid at 25° C.
(D) a silicone oil having a kinematic viscosity at 25° C. of 100 cSt or less
Condition (1): (A)/((B)+(C)+(D))=0.01 to 1.0
Condition (2): (A)/(C)=00.1 to 1.0
Condition (3): viscosity at 25° C. being 10 to 100000 mPa·s.

2. The emulsion cosmetic according to claim 1, wherein the component (A) is one or two or more types selected from stearoyl methyl taurate, cocoyl methyl taurate and lauroyl methyl taurate.

3. The emulsion cosmetic according to claim 1, wherein the emulsion cosmetic further comprises a silicone surfactant (E).

4. The emulsion cosmetic according to claim 1, wherein the emulsion cosmetic further comprises 0.05 to 2.0% by mass of the silicone surfactant (E) in the cosmetic.

5. The emulsion cosmetic according to claim 1, wherein the component (D) has a phenyl group in a molecule.

6. The emulsion cosmetic according to claim 1, wherein 0.1 to 20% by mass of the component (D) is comprised in the cosmetic.

7. The emulsion cosmetic according to claim 1, wherein the emulsion cosmetic comprises a polyol or sugar alcohol having Log P of −1.0 to −3.0.

8. The emulsion cosmetic according to claim 1, wherein the emulsion cosmetic comprises an ester oil or an animal/vegetable oil having an IOB value in an organic conceptual diagram of 0 to 0.2.

9. The emulsion cosmetic according to claim 1, wherein the emulsion cosmetic comprises an ester oil that has an IOB value in an organic conceptual diagram of 0 to 0.2 and is liquid at 25° C.

10. The emulsion cosmetic according to claim 1, wherein the component (C) is a C₁₀₋₂₄ aliphatic alcohol having a linear or branched chain.

11. The emulsion cosmetic according to claim 1, wherein the component (C) is behenyl alcohol.

12. The emulsion cosmetic according to claim 1, wherein the component (E) is a polyglyceryl modified silicone.

13. The emulsion cosmetic according to claim 1, wherein the component (E) is a polyether (3-40) modified polysiloxane.

14. The emulsion cosmetic according to claim 1, wherein the emulsion cosmetic further comprises a water-soluble thickener (F).

15. The emulsion cosmetic according to claim 1, wherein the emulsion cosmetic comprises a semi low-soluble or low-soluble drug having a Log P value of 10 to 30.

16. The emulsion cosmetic according to claim 1, wherein the semi low-soluble or low-soluble drug having a Log P value of 10 to 30 is selected from Coenzyme Q10, stearyl glycyrrhetinate, astaxanthin, tranexamic acid, allantoin, alkoxy salicylic acid and a whitening agent (other components).

17. The emulsion cosmetic according to claim 1, wherein the component (B) is dimethicone/phenyl vinyl dimethicone crosspolymer, polysilicone-11 or a crosslinked methylpolysiloxane.
